(19) United States
(12) Patent Application Publication
(10) Pub. No.: US 2021/0368887 A1
(43) Pub. Date: Dec. 2, 2021

(54) FACE SHIELDS WITH SHADOW-REDUCTION MOUNTS

(71) Applicant: Ronald C. NGUYEN, Fountain Valley, CA (US)

(72) Inventor: Ronald C. NGUYEN, Fountain Valley, CA (US)

(21) Appl. No.: 17/243,557

(22) Filed: Apr. 28, 2021

Related U.S. Application Data
(60) Provisional application No. 63/016,941, filed on Apr. 28, 2020.

(51) Int. Cl. A41D 13/11; A41D 1/00

(52) U.S. Cl. CPC A41D 13/1184; A41D 1/002

(57) ABSTRACT

Face shield assemblies for protecting a face of a user are described. The face shield assemblies may be configured for use with a light. In one embodiment, a face shield assembly comprises a face shield having a front shield. The face shield assembly may also comprise a shadow-reduction mount coupled to the face shield. The shadow-reduction mount may have an arm. The shadow-reduction mount may also have a light mount coupled to an end portion of the arm. The light mount may be configured to be coupled to the light. The shadow-reduction mount may be configured to position the light at or below an eye level of the user.

### CROSS-REFERENCE TO RELATED APPLICATION

This application claims the benefit of U.S. provisional patent application Ser. No. 63/016,941, filed Apr. 28, 2020, which is hereby incorporated by reference in its entirety.

### BACKGROUND

Face shields may be worn by a user to protect the face. However, when the user is also wearing a loupe light or other eyewear-mounted light, the light may create a glare from the reflection off the inside of the face shield.

To eliminate the glare, the light may be mounted to the face shield, such as to a visor of the face shield. However, mounting the light on the visor or other part of the face shield may cause or worsen shadows when illuminating an area of interest, especially narrow areas such as holes.

What is needed is a face shield which may be used with a light to reduce shadows when illuminating an area of interest.

### SUMMARY

Face shield assemblies for protecting a face of a user are described. The face shield assemblies may be configured for use with a light. In one embodiment, a face shield assembly comprises a face shield having a front shield. The face shield assembly may also comprise a shadow-reduction mount coupled to the face shield. The shadow-reduction mount may have an arm. The shadow-reduction mount may also have a light mount coupled to an end portion of the arm. The light mount may be configured to be coupled to the light. The shadow-reduction mount may be configured to position the light at or below an eye level of the user.

Shadow-reduction mounts for coupling a light to an accessory mount of a face shield are described. In one embodiment, a shadow-reduction mount may comprise an arm. The shadow-reduction mount may also comprise a bracket coupled to a first end portion of the arm. The bracket may be configured to be coupled to the accessory mount of the face shield. The shadow-reduction mount may also comprise a light mount coupled to a second end portion of the arm. The light mount may be configured to be coupled to the light. The arm may be configured to position the light at or below an eye level of a user of the face shield.

### BRIEF DESCRIPTION OF THE DRAWINGS

FIG. 1A shows one embodiment of a face shield assembly 1000, including a face shield 1100 coupled to a light 1200 by a shadow-reduction mount 1300.

FIG. 1B shows one embodiment of a face shield 1100.

FIGS. 1C-1D show one embodiment of a shadow-reduction mount 1300 coupled to a face shield 1100. FIG. 1E-1F show front and rear views of one embodiment of a shadow-reduction mount 1300.

FIG. 2A shows a side view of a face shield assembly 1000 as worn by a user, showing a line of sight. FIG. 2B shows a top view of a face shield assembly 1000 as worn by a user, showing lines of sight for both eyes.

### DESCRIPTION

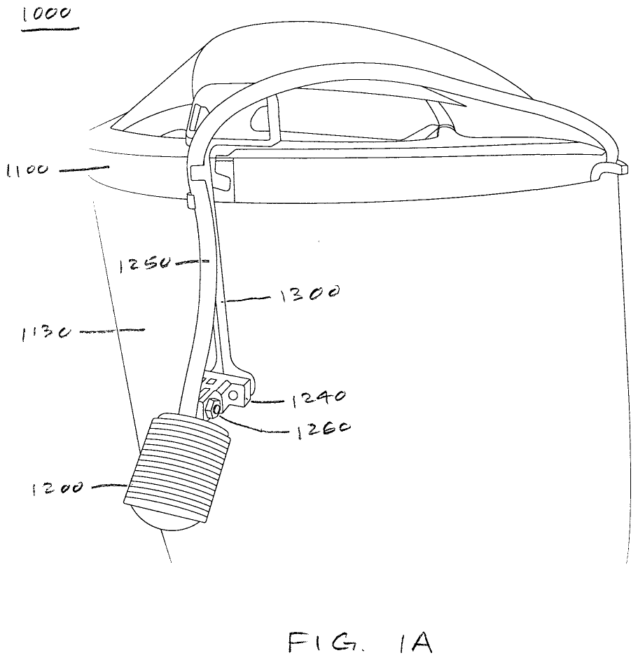

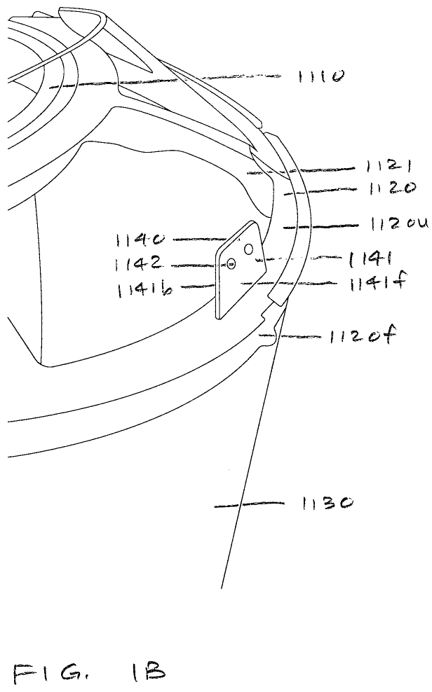

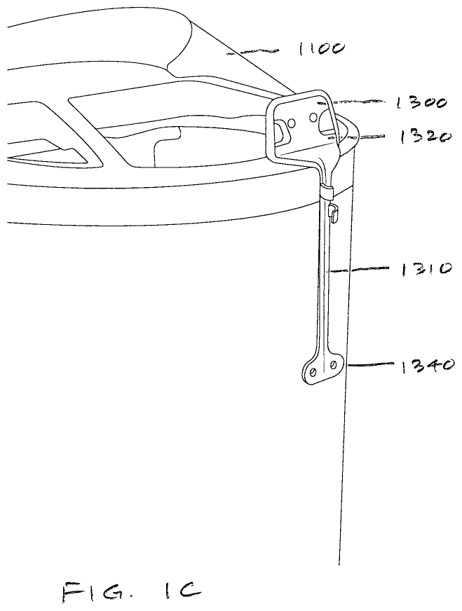

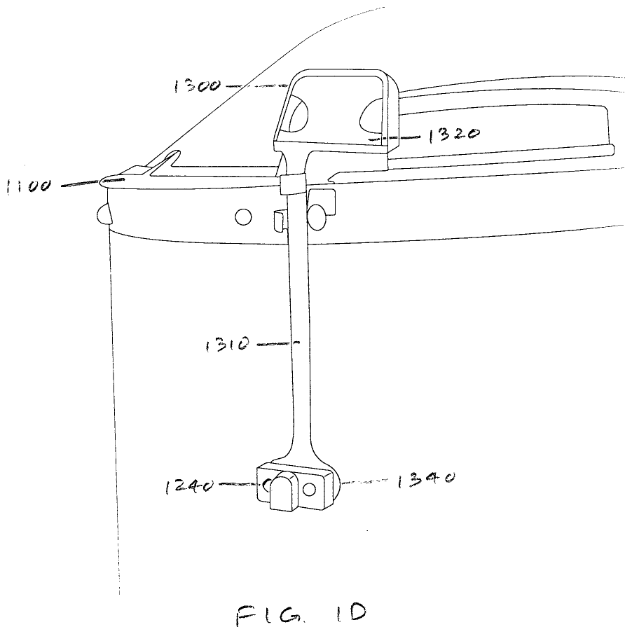

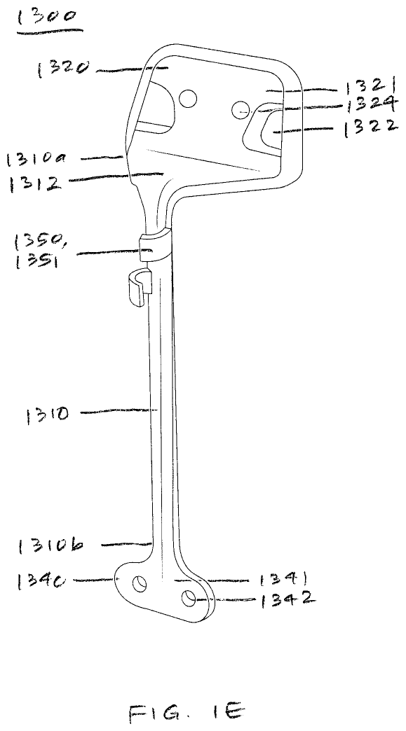

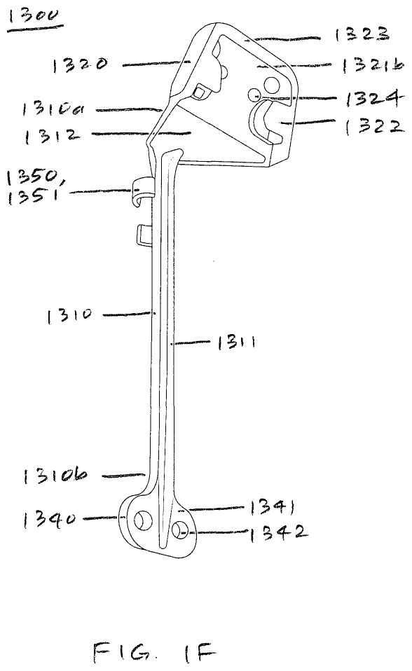

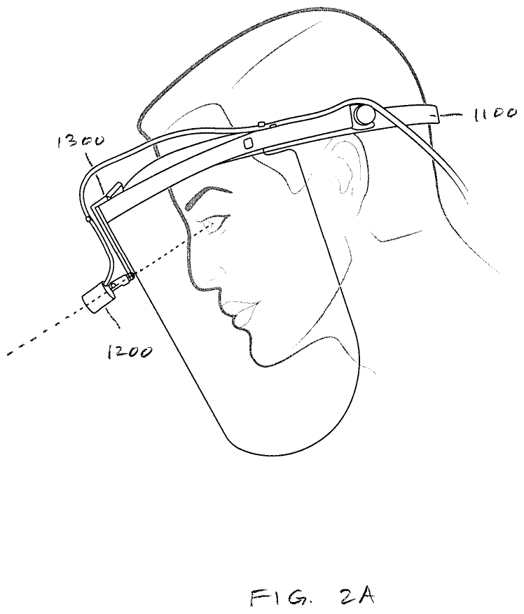

FIG. 1A shows one embodiment of a face shield assembly 1000, including a face shield 1100 coupled to a light 1200 by a shadow-reduction mount 1300. FIG. 1B shows one embodiment of a face shield 1100.

Face shield assembly 1000 may include a face shield 1100. Face shield 1100 may be worn by a user to protect the face.

Face shield 1100 may include a headband 1110. Headband 1110 may be worn on and/or around the head. Headband 1110 may be adjustable to accommodate heads of different sizes and to provide a proper fit.

Face shield 1100 may include a visor 1120. Visor 1120 may be coupled to headband 1110. Visor 1120 may include an upper surface 1120u and a front surface 1120f. Visor 1120 may be fixed or may be able to flip up and down.

Visor 1120 may include a top shield 1121. Top shield 1121 may be coupled to upper surface 1120u and/or any other part of visor 1120. Top shield 1121 may protect the face of the user from above through visor 1120.

Face shield 1100 may include a front shield 1130. Front shield 1130 may be coupled to front surface 1120f of visor 1120 and/or any other part of face shield 1100. Front shield 1130 may protect the face of the user.

Visor 1120 may extend from the face. Visor 1120 may extend from the front of the head. Visor 1120 may provide space behind front shield 1130 for the face of the user. Visor 1120 may provide space behind front shield 1130 for loupes, eyewear, masks, and other items worn on the face of the user.

Face shield 1100 may include an accessory mount 1140. Accessory mount 1140 may be coupled to upper surface 1120u of visor 1120 and/or any other part of face shield 1100. Accessory mount 1140 may be coupled to a center of visor 1120 and/or a center of any other part of face shield 1100. Accessory mount 1140 may be located along a midline of the face of the user. Accessory mount 1140 may allow one or more accessories, such as a light, to be coupled to face shield 1100.

Accessory mount 1140 may include a tab 1141. Tab 1141 may be coupled to upper surface 1120u of visor 1120 and/or any other part of face shield 1100. Tab 1141 may include a front surface 1141f and a back surface 1141b. Tab 1141 may have a shape that is trapezoidal, rectangular, or any other suitable shape. Tab 1141 may be vertical or angled.

Accessory mount 1140 may include one or more holes 1142. Holes 1142 may be formed in tab 1141 and/or any other part of face shield 1100. Holes 1142 may allow bolts and/or other fasteners to couple an accessory, such as a light, to face shield 1100.

Face shield assembly 1000 may include a light 1200. Light 1200 may include a loupe light and/or any other suitable light. Light 1200 may provide light to an area of interest.

Light 1200 may include an adaptor block 1240. Adaptor block 1240 may be configured to couple light to accessory mount 1140 or other mount.

Light 1200 may include a light wire 1250. Light wire 1250 may carry power and/or control signals to light 1200.

Light 1200 may include a hinge 1260. Hinge 1260 may allow light 1200 to be directed at a point of interest.

FIGS. 1C-1D show one embodiment of a shadow-reduction mount 1300 coupled to a face shield 1100. FIG. 1E-1F show front and rear views of one embodiment of a shadow-reduction mount 1300.

Face shield assembly 1000 may include a shadow-reduction mount 1300.

Shadow-reduction mount 1300 may include at least one arm 1310. Arm 1310 may include first end portion 1310a and a second end portion 1310b. Arm 1310 may have a width and/or thickness that is uniform or non-uniform. Arm 1310 may have a length that is adjustable. Arm 1310 may be available in a plurality of lengths. Arm 1310 may have a length of approximately one inch to approximately four inches. Arm 1310 may include at least one rib 1311. Rib 1311 may reinforce arm 1310.

Arm 1310 or a portion of arm 1310 may be configured to be positioned in front of front shield 1130. Arm 1310 or a portion of arm 1310 may be configured to extend down the front of front shield 1130. Arm 1310 or a portion of arm 1310 may be configured to be positioned close to or against the front of the front shield 1130. The portion of arm 1310 that is positioned in a field of view of the user may have a width that is narrow to reduce the visual profile of arm 1310 to the user.

Arm 1310 may include a clearance segment 1312. Clearance segment 1312 may provide enough clearance at front surface 1120f of visor 1120 for front shield 1130 to be removed and replaced.

Shadow-reduction mount 1300 may include a bracket 1320. Bracket 1320 may be coupled to first end portion 1310a of arm 1310. Bracket 1320 may be configured to be coupled to accessory mount 1140 of face shield 1100.

Bracket 1320 may include a bracket plate 1321. Bracket plate 1321 may have a shape that is trapezoidal, rectangular, or any other suitable shape. Bracket plate 1321 may have a shape similar to that of tab 1141. Bracket plate 1321 may include a back surface 1321b. Back surface 1321b of bracket plate 1321 may be configured to be coupled against front surface 1141f of tab 1141.

Bracket 1320 may include one or more bracket clips 1322. Bracket clips 1322 may be coupled to bracket plate 1321 and/or any other part of bracket 1320. Bracket clips 1322 may be configured to clip to and/or at least partially around tab 1141 of accessory mount 1140. Bracket clips 1322 may be configured to clip against back surface 1141b of tab 1141.

Bracket plate 1321 may include a lip 1323. Lip 1323 may be formed on at least a portion of an edge of bracket plate 1321. Lip 1323 may be configured to be at least partially seated against an edge of tab 1141. Bracket clips 1322 may be coupled to lip 1323.

Bracket plate 1321 may include one or more pips 1324. Pips 1324 may be formed on back surface 1321b of bracket plate 1321. Pips 1324 may be configured to line up with holes 1142 of accessory mount 1140 when bracket 1320 is coupled to accessory mount 1140. Pips 1324 and holes 1142 may cause audible and/or tactile feedback as bracket 1320 is coupled to accessory mount 1140.

Shadow-reduction mount 1300 may include a light mount 1340. Light mount 1340 may be coupled to second end portion 1310b of arm 1310. Light mount 1340 may allow one or more accessories, such as light 1200, to be coupled to shadow-reduction mount 1300.

Light mount 1340 may include a plate 1341. Light mount 1340 may include one or more holes 1342. Holes 1342 may be formed in plate 1341 and/or any other part of light mount 1340. Holes 1342 may allow bolts and/or other fasteners to couple an accessory, such as light 1200, to shadow-reduction mount 1300.

Arm 1310 may be configured to position light 1200 at or below an eye level of the user. Arm 1310 may be configured to position light 1200 at or below an eye level of the user when arm 1310 is coupled to face shield 1100, and when face shield 1100 is worn by the user.

Arm 1310 may be configured to position light 1200 in front of the face of the user. Arm 1310 may be configured to position light 1200 along a midline of the face of the user. Arm 1310 may be configured to position light 1200 in front of front shield 1130. Arm 1310 may be configured to position light 1200 in a same plane or at a same level with lines of sight for both eyes, when both eyes are looking at the same point of interest, for a point of interest that is at or below eye level. A line of sight may be an imaginary line between the center of a pupil of an eye, and a point of interest. Arm 1310 may be configured to position light 1200 along a line between a point halfway between the eyes and a point of interest. Arm 1310 may thus reduce shadows when light 1200 is used to illuminate an area of interest, especially narrow areas such as holes.

Shadow-reduction mount 1300 may include wire management 1350. Wire management 1350 may be coupled to arm 1310 and/or any other part of shadow-reduction mount 1300. Wire management 1350 may couple light wire 1250 to shadow-reduction mount 1300. Wire management 1350 may reduce the likelihood of light wire 1250 of becoming entangled with or caught on another piece of equipment. Wire management 1350 may include one or more wire clips 1351.

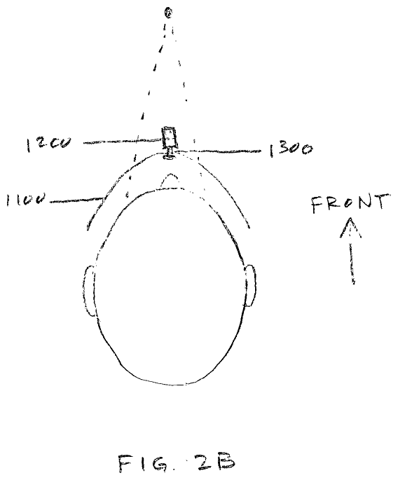

FIG. 2A shows a side view of a face shield assembly 1000 as worn by a user, showing a line of sight. FIG. 2B shows a top view of a face shield assembly 1000 as worn by a user, showing lines of sight for both eyes.

Shadow-reduction mount 1300 may be configured to position light 1200 at or below an eye level of the user. Shadow-reduction mount 1300 may be configured to position light 1200 at or below an eye level of the user when shadow-reduction mount 1300 is coupled to face shield 1100, and when face shield 1100 is worn by the user.

Shadow-reduction mount 1300 may be configured to position light 1200 in front of the face of the user. Shadow-reduction mount 1300 may be configured to position light 1200 along a midline of the face of the user. Shadow-reduction mount 1300 may be configured to position light 1200 in front of front shield 1130. Shadow-reduction mount 1300 may be configured to position light 1200 in a same plane or at a same level with lines of sight for both eyes, when both eyes are looking at the same point of interest, for a point of interest that is at or below eye level. A line of sight may be an imaginary line between the center of a pupil of an eye, and a point of interest. Shadow-reduction mount 1300 may be configured to position light 1200 along a line between a point halfway between the eyes and a point of interest. Shadow-reduction mount 1300 may thus reduce shadows when light 1200 is used to illuminate an area of interest, especially narrow areas such as holes.

Light 1200 and shadow-reduction mount 1300 may be positioned in or near the center of the direct field of view of the user; however, when the user is focused on a point of interest beyond light 1200, the user tends to become less distracted over time by light 1200 and shadow-reduction mount 1300. Also, because each eye has an unobstructed line of sight to the point of interest, the point of interest may still be seen clearly by the user. In addition, when a user is wearing loupes, light 1200 and shadow-reduction mount 1300 may not be visible at all in the field of view of the loupes.

1. A face shield assembly for protecting a face of a user, the face shield assembly configured for use with a light, the face shield assembly comprising:
a face shield having a front shield; and
a shadow-reduction mount coupled to the face shield, the shadow-reduction mount having an arm, the shadow-reduction mount having a light mount coupled to an end portion of the arm, the light mount configured to be coupled to the light;
wherein the shadow-reduction mount is configured to position the light at or below an eye level of the user.

2. The face shield assembly of claim 1, wherein the shadow-reduction mount is configured to position the light in front of the face.

3. The face shield assembly of claim 1, wherein the shadow-reduction mount is configured to position the light along a midline of the face.

4. The face shield assembly of claim 1, wherein the shadow-reduction mount is configured to position the light in front of the front shield.

5. The face shield assembly of claim 1, wherein the shadow-reduction mount is configured to position the light at a same level with lines of sight for both eyes of the user.

6. The face shield assembly of claim 1, wherein at least a portion of the arm is configured to be positioned in front of the front shield.

7. A shadow-reduction mount for coupling a light to an accessory mount of a face shield, the shadow-reduction mount comprising:
an arm;
a bracket coupled to a first end portion of the arm, the bracket configured to be coupled to the accessory mount of the face shield; and
a light mount coupled to a second end portion of the arm, the light mount configured to be coupled to the light;
wherein the arm is configured to position the light at or below an eye level of a user of the face shield.

8. The shadow-reduction mount of claim 7, wherein the arm is configured to position the light in front of a face of the user.

9. The shadow-reduction mount of claim 7, wherein the arm is configured to position the light along a midline of a face of the user.

10. The shadow-reduction mount of claim 7, wherein the arm is configured to position the light in front of a front shield of the face shield.

11. The shadow-reduction mount of claim 7, wherein the arm is configured to position the light at a same level with lines of sight for both eyes of the user.

12. The shadow-reduction mount of claim 7, wherein at least a portion of the arm is configured to be positioned in front of a front shield of the face shield.

13. The shadow-reduction mount of claim 7, wherein the bracket includes at least one bracket clip coupled to the first end portion of the arm, the bracket clip configured to be coupled to the accessory mount.

14. The shadow-reduction mount of claim 7, wherein the bracket includes a bracket plate coupled to the first end portion of the arm.

15. The shadow-reduction mount of claim 7, wherein the light mount includes a plate coupled to the second end portion of the arm.

16. The shadow-reduction mount of claim 7, wherein the light mount includes one or holes formed in the second end portion of the arm.

17. A face shield assembly for protecting a face of a user, the face shield assembly configured for use with a light, the face shield assembly comprising:
a face shield having a front shield; and
a light positioning means coupled to the face shield, the light positioning means having an arm, the light positioning means having a light mount coupled to an end portion of the arm, the light mount configured to be coupled to the light;
wherein the light positioning means is configured to position the light at or below an eye level of the user.

18-23. (canceled)
